(19) United States
(12) Patent Application Publication
(10) Pub. No.: US 2013/0231659 A1
(43) Pub. Date: Sep. 5, 2013

(54) OFF-WALL AND CONTACT ELECTRODE DEVICES AND METHODS FOR NERVE MODULATION

(71) Applicant: BOSTON SCIENTIFIC SCIMED, INC., Maple Grove, MN (US)

(72) Inventors: JASON P. HILL, BROOKLYN PARK, MN (US); MARTIN R. WILLARD, BURNSVILLE, MN (US)

(73) Assignee: BOSTON SCIENTIFIC SCIMED, INC., MAPLE GROVE, MN (US)

(21) Appl. No.: 13/783,049

(22) Filed: Mar. 1, 2013

Related U.S. Application Data
(60) Provisional application No. 61/605,649, filed on Mar. 1, 2012.

(51) Int. Cl. A61B 18/14

(52) U.S. Cl. CPC A61B 18/1492
USPC 606/41

(57) ABSTRACT

Medical devices and methods for making and using medical devices are disclosed. An example medical device may include a system for nerve modulation. The system may include an elongate shaft and a nerve modulation assembly disposed at the distal end of the shaft. The nerve modulation assembly may have a collapsed configuration and an expanded configuration. The nerve modulation assembly may include an inner basket and an outer basket. The inner basket may include a plurality of electrode struts. Each electrode strut may include an electrode. The outer basket may include a plurality of spacer struts.

### CROSS-REFERENCE TO RELATED APPLICATIONS

This application claims priority under 35 U.S.C. §119 to U.S. Provisional Application Ser. No. 61/605,649, filed Mar. 1, 2012, the entirety of which is incorporated herein by reference.

### TECHNICAL FIELD

The present disclosure pertains to medical devices, and methods for manufacturing medical devices. More particularly, the present disclosure pertains to methods and apparatuses for modulating nerves through the walls of blood vessels.

### BACKGROUND

Certain treatments require temporary or permanent interruption or modification of select nerve functions. One example treatment is renal nerve ablation, which is sometimes used to treat conditions related to congestive heart failure. The kidneys produce a sympathetic response to congestive heart failure, which among other effects, increases the undesired retention of water and/or sodium. Ablating some nerves running to the kidneys may reduce or eliminate this sympathetic function, providing a corresponding reduction in the associated undesired symptoms. For example, a renal nerve ablation procedure is often used to lower the blood pressure of hypertensive patients.

Many nerves (and nervous tissue such as brain tissue), including renal nerves, run along the walls of or in close proximity to blood vessels and these nerves can be accessed intravascularly through the blood vessel walls. In some instances, it may be desirable to ablate or otherwise modulate perivascular renal nerves using a radio frequency (RF) electrode. Such treatment, however, may result in thermal injury to the vessel at the electrode and other undesirable side effects such as, but not limited to, blood damage, clotting, and/or protein fouling of the electrode. To prevent such undesirable side effects, some techniques attempt to increase the distance between the vessel walls and the electrode. In these systems, however, the electrode may inadvertently contact the vessel walls.

Therefore, there remains room for improvement and/or alternatives in providing systems and methods for intravascular nerve modulation.

### SUMMARY

The disclosure is directed to several alternative designs and methods of using medical device structures and assemblies.

Accordingly, some embodiments pertain to a system for nerve modulation, including an elongate shaft having a proximal end, a distal end, and a nerve modulation assembly at the distal end. The nerve modulation assembly has a collapsed configuration and an expanded configuration. The system may further include an inner basket having a proximal end and a distal end and multiple electrode struts joined to each other at the proximal end of the inner basket and extending to the distal end of the inner basket. Each electrode strut includes an electrode. The electrodes may be monopolar or bipolar. The electrodes of the system may be powered with a single power controller for all electrodes or use dedicated power controllers for each electrode. The power to the electrodes might be delivered simultaneously to all electrodes or in some sequential pattern. In addition, an outer basket having a proximal end and a distal end and a plurality of spacer struts joined to each other at the proximal end of the outer basket and extending to the distal end of the outer basket. The inner basket and the outer basket are disposed at the distal end of the elongate shaft, wherein in the expanded configuration, the plurality of spacer struts extend further radially from the elongate axis than the plurality of electrode struts.

An example system for nerve modulation may include an elongate shaft having a longitudinal axis, a proximal end, a distal end, and a nerve modulation assembly disposed at the distal end. The nerve modulation assembly may have a collapsed configuration and an expanded configuration. The nerve modulation assembly may include an inner basket and an outer basket. The inner basket may include a proximal end and a distal end. The inner basket may also include a plurality of electrode struts joined to each other at the proximal end of the inner basket and extending to the distal end of the inner basket. Each electrode strut may include an electrode. The outer basket may include a proximal end and a distal end. The outer basket may also include a plurality of spacer struts joined to each other at the proximal end of the outer basket and extending to the distal end of the outer basket. The inner basket and the outer basket may be disposed at the distal end of the elongate shaft. When the nerve modulation assembly is in the expanded configuration the plurality of spacer struts may extend further radially outward from the longitudinal axis of the shaft than the plurality of electrode struts.

Another example system for nerve modulation may include an elongate shaft having a proximal end, a distal end and a nerve modulation assembly at the distal end. The nerve modulation assembly may include a basket configured to shift between a collapsed configuration and an expanded configuration. The basket may have a proximal end and a distal end. The basket may include a plurality of inner struts and a plurality of outer struts. Each of the inner struts may include an electrode portion and an electrically insulated portion. The basket may be disposed at the distal end of the elongate shaft.

Another example system for nerve modulation may include an elongate shaft having a proximal end and a distal end. A basket assembly configured to move between a collapsed configuration and an expanded configuration may be disposed adjacent to the distal end of the elongate shaft. The basket assembly may include an inner basket having a proximal end and a distal end and comprising a first plurality of struts. At least one of the first plurality struts may include an electrode. The basket assembly may also include an outer basket having a proximal end and a distal end and comprising a second plurality of struts. The second plurality of struts may include an insulating material. In the expanded configuration the outer basket may have a cross-sectional profile larger than a cross-sectional profile of the inner basket.

The summary of some example embodiments is not intended to describe each disclosed embodiment or every implementation of the disclosure.

### BRIEF DESCRIPTION OF THE DRAWINGS

The present disclosure may be more completely understood in consideration of the following detailed description of various embodiments in connection with the accompanying drawings, in which:

FIG. 1 is a schematic view illustrating a renal nerve modulation system in situ.

FIG. 2A is a schematic view of an exemplary ablative catheter system with an ablative member in the expanded state.

FIG. 2B illustrates the ablative member of FIG. 2A in a collapsed position.

FIG. 3A illustrates the distal end of an exemplary ablative catheter system in an expanded position within a blood vessel.

FIG. 3B is a cut away sectional view of the ablative member of FIG. 2A.

FIG. 4 illustrates the distal end of an alternate ablative catheter system in an expanded position within a blood vessel.

FIG. 5 is cross-sectional view of an embodiment of an ablative catheter system with an ablative member in an expanded state within a blood vessel.

FIG. 6 is cross-sectional view of an embodiment of an ablative catheter system with an ablative member in an expanded state within a blood vessel.

FIG. 7 is an isometric view of the distal portion of an example ablative catheter system with an ablative member in the expanded state.

FIG. 8 is an isometric view of the distal portion of an example ablative catheter system with an ablative member in the expanded state.

FIG. 9 is an isometric view of the distal portion of an example ablative catheter system with an ablative member in the expanded state.

FIG. 10A is an isometric view of the distal portion of an example ablative catheter system with an ablative member in the expanded state.

FIG. 10B is an end view of the distal portion of the example ablative catheter system of FIG. 10A with an ablative member in the expanded state.

FIGS. 11A and 11B are isometric views of the distal portion of an ablative catheter system shown in an expanded state and a collapsed state, respectively.

FIG. 12 is an isometric view of the distal portion of an ablative catheter system shown in an expanded state.

FIG. 13 is an isometric view of the distal portion of an ablative catheter system shown in an expanded state.

FIG. 14 is an isometric view of the distal portion of an ablative catheter system shown in an expanded state.

While embodiments of the present disclosure are amenable to various modifications and alternative forms, specifics thereof have been shown by way of example in the drawings and will be described in detail. It should be understood, however, that the intention is not to limit aspects of the disclosure to the particular embodiments described. One the contrary, the intention is to cover all modifications, equivalents, and alternatives falling within the spirit and scope of the present disclosure.

### DETAILED DESCRIPTION

For the following defined terms, these definitions shall be applied, unless a different definition is given in the claims or elsewhere in the specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimension ranges and/or values pertaining to various components, features, and/or specifications are disclosed, one of skill in the art, incited by the present disclosure, would understand desired dimensions, ranges and/or values many deviate from those expressly disclosed.

As used in this specification and the appended claims, the singular forms “a,” “an,” and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the disclosure. The illustrative embodiments depicted are intended only as exemplary. Selected features of any illustrative embodiment may be incorporated into an additional embodiment unless clearly stated to the contrary.

While the devices and methods described herein are discussed relative to renal nerve modulation, it is contemplated that the devices and methods may be used in other applications where ablation or modulation are desired such as nerve modulation and/or ablation near other vessel lumens.

In some instances, it may be desirable to ablate perivascular renal nerves with targeted tissue heating. However, as energy passes from an electrode to the desired treatment region the energy may heat the fluid (e.g. blood) and tissue as it passes. As more energy is used, higher temperatures in the desired treatment region may be achieved, but may result in some negative side effects, such as, but not limited to, thermal injury to the vessel wall, blood damage, clotting, and/or electrode fouling. Positioning the electrode away from the vessel wall may provide some degree of passive cooling by allowing blood to flow past the electrode while still allowing the electrode elements to target nerves within about 2.5 mm of the luminal surface, where the perivascular renal nerves are located. An appropriate amount of energy may properly ablate the nerve tissue while causing no damage to the vessel wall or to deep tissue such as muscle tissue or the intestinal walls.

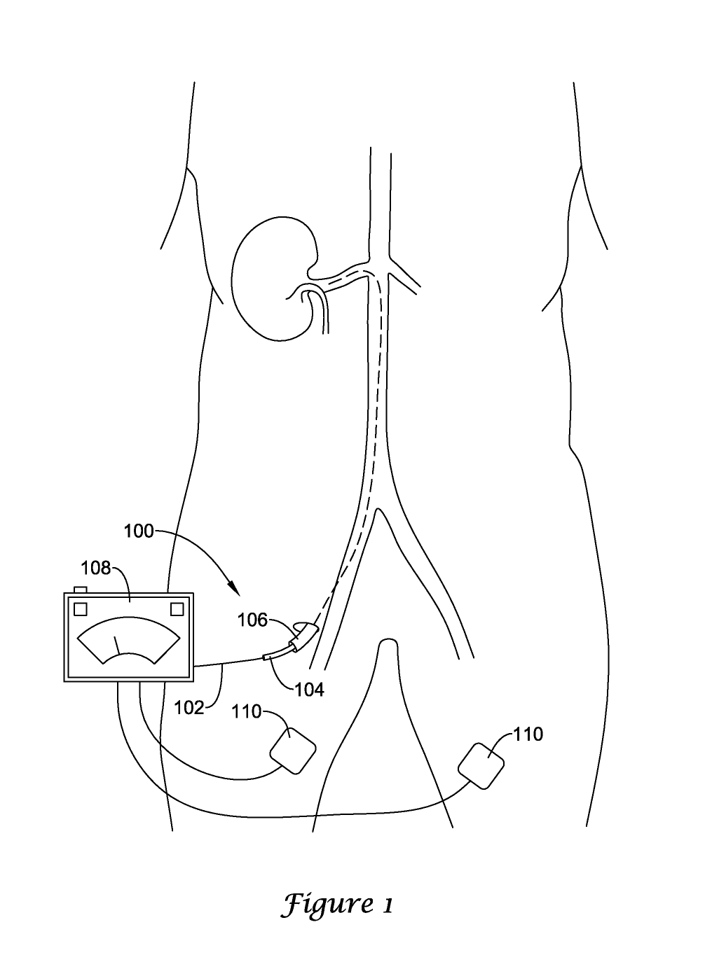

FIG. 1 is a schematic view of an illustrative renal nerve modulation system 100 in situ. System 100 may include one or more conductors 102 for providing power to a nerve modulation assembly 104 disposed within a catheter sheath or guide catheter 106. A proximal end of the conductor 102 may be connected to a control and power element 108, which supplies the necessary electrical energy to activate the one or more electrodes (not shown) at or near a distal end of the nerve modulation assembly 104. In some instances, return electrode patches 110 may be supplied on the struts or at another conventional location on the patient's body to complete the circuit. In bipolar designs, the ground electrodes may be present on the device near the distal end. The control and power element 108 may include monitoring elements to monitor parameters such as power, temperature, voltage, amperage, impedance, pulse size and/or shape and other suitable parameters as well as suitable controls for performing the desired procedure. In some instances, the power element 108 may control a radio frequency (RF) electrode. The electrode may be configured to operate at a frequency of approximately 460 kHz. It is contemplated that any desired frequency in the RF range may be used, such as, for example, from 400-900 kHz. However, it is contemplated that different types of energy outside the RF spectrum may be used as desired, such as, for example, but not limited to ultrasound, microwave, and laser.

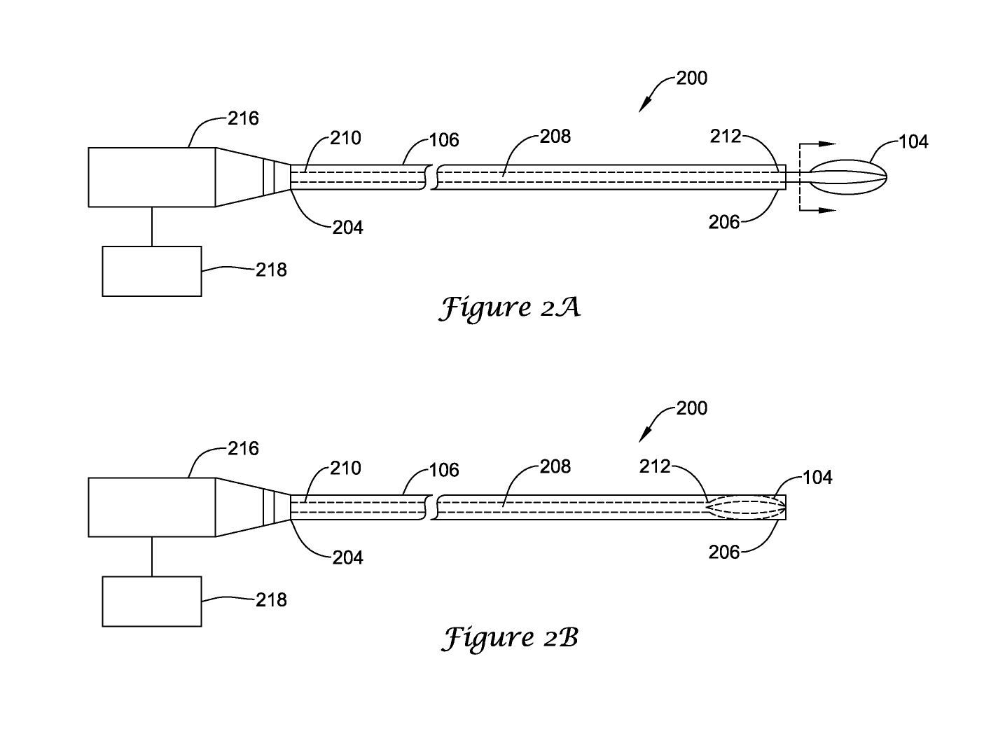

FIGS. 2A and 2B are schematics of an exemplary ablative catheter system 200 according to embodiments of the present disclosure. More particularly, FIG. 2A is a side view of the catheter system 200 in an expanded state, while FIG. 2B is a side view of the catheter system 200 in a collapsed or compressed state. The ablative catheter system 200 includes catheter sheath 106 having a proximal end 204 and a distal end 206, an elongate member 208 having a proximal end 210 and a distal end 212, and an expandable ablative member, such as the nerve modulation assembly 104 coupled to the elongate member's distal end 212. The catheter system 200 may further include a handle 216 coupled to the sheath's proximal end 204.

The sheath 106 may be substantially circular, formed of any suitable biocompatible material such as polyurethane, polyether block amide, polyimide, nylon, polyester, polyethylene, or any other such polymeric materials. The sheath 106 may also be a composite structure comprising a polymer matrix and a braid that is also a polymer or metal. Other suitable cross-sectional shapes such as elliptical, oval, polygonal, or irregular may also be contemplated. Moreover, the sheath 106 may be flexible along its entire length or adapted for flexure along portions of its length. Alternatively, the sheath's distal end 206 may be flexible while the remaining sheath may be rigid. Flexibility allows the sheath 106 to maneuver in the circuitous vasculature, while rigidity provides the necessary rigidity to allow the operator to urge the sheath 106 forward. The diameter of the sheath 106 may vary according to the desired application, but it is generally smaller than the typical diameter of a patient's vasculature. Moreover, the diameter of the sheath 106 may depend on the diameter of the elongate member 208 and the nerve modulation assembly 104.

The elongate member 208, as described previously, extends along the elongate axis from the proximal end 204 of the sheath 106. Further, the elongate member's proximal end 210 may be connected to the handle 216 and its distal end 212 may be connected to the nerve modulation assembly 104. The connection to the handle 216 and the nerve modulation assembly 104 may be temporary or permanent. Examples of temporary connection include snap-fit, Luer-lock, or screw-fit devices. Examples of permanent or semi-permanent connection include welding or gluing. It will be understood that various other connection mechanisms may be incorporated to connect the various members. In other instances, the elongate member 208 may not be connected to the handle 216. Instead, the handle 216 may include one or more ports (not shown) and the elongate member 208 may be inserted in the catheter sheath's lumen through the port. Using an independent elongate member 208 and nerve modulation assembly 104 allows operators to use the catheter sheath 106 for other procedures or to insert guidewires for guiding and urging the catheter to the desired location.

In one embodiment, the elongate member 208 is a conductor covered by an insulative material. The proximal end of the conductor may be connected to a power source 218 such as an external power generator or battery incorporated in the handle 216. The distal end of the conductor may be connected to the nerve modulation assembly 104.

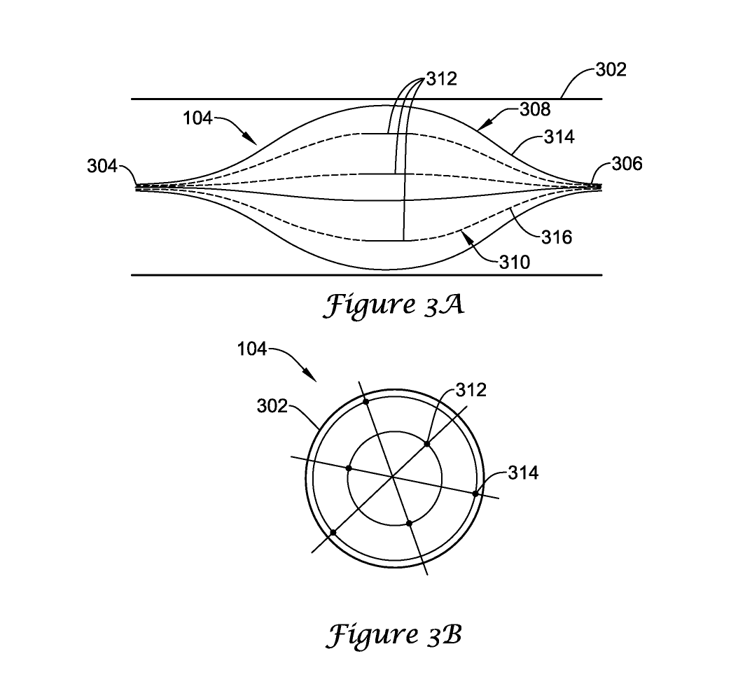

FIG. 2A illustrates the nerve modulation assembly 104 in an expanded state. In general, the nerve modulation assembly 104 is configured as a bi-level basket having an outer basket that contacts the blood vessel walls and an inner basket that includes electrodes for ablation purposes. Electrodes positioned on the inner basket of the nerve modulation assembly 104 remain spaced from the vessel wall. Depending on the desired application, electrodes may be placed in any desired position on the inner basket of the nerve modulation assembly 104. The nerve modulation assembly 104 is discussed in detail in the following section in connection with FIGS. 3A and 3B.

FIG. 2B is a schematic illustrating the distal portion of the ablative catheter system 200 with the nerve modulation assembly 104 in the compressed state. From this state, the ablative member may be expanded using numerous techniques depending on the properties of the ablative member. These techniques may be applied on each of the inner and outer basket, expanding the baskets to the desired degree. For instance, the ablative member 104 may be self-expandable or expanded by some external force such as a pull wire. Self-expandable members may be formed of any material that is in a compressed state when force is applied and in an expanded state when force is released. Such members may be formed of steel or of shape memory alloys such as Nitinol or any other self-expandable material.

Many techniques may be utilized to compress a self-expandable member and keep it in the compressed state. According to one technique, the nerve modulation assembly 104 is present within the sheath 106 for deployment (shown in FIG. 2B). The inner diameter of sheath 106 is smaller than the expanded state of nerve modulation assembly 104, keeping it in the compressed state. Once the assembly 104 exits the sheath 106, however, the pressure is released, and the modulation assembly 104 expands. It will be understood that in such situations, the material and thickness of the sheath 106 is selected such that it applies a greater force on the nerve modulation assembly 104 than the force exerted by the modulation assembly 104 on the sheath 106. If the sheath 106 material is too thin or too elastic, it may not be sufficient to hold the nerve modulation assembly 104 in the compressed state, and the nerve modulation assembly 104 may expand within the sheath 106 itself. Alternatively, if the sheath 106 is too rigid or thick, it may not be able to traverse the circuitous vasculature path, causing injury to the vessel walls. Therefore, it may be often preferred to select a suitable material and thickness keeping both aspects in mind.

According to another technique, pull wires (not shown) may be utilized. Pull wires may be attached to the ablative member's distal end or proximal end. In some instances, pull wires may be connected to both the inner and the outer basket. This may allow a user to selectively control the configuration of each basket individually. When the pull wire is pulled in a certain axial direction (distally or proximally), it places a tensile force on the nerve modulation assembly 104, stretching it longitudinally and keeping it in the compressed state. When the pull wire is released, the tensile force is released permitting the nerve modulation assembly 104 to enter the expanded state. For example, if the pull wire is attached to the ablative member's distal end, pulling the wire distally elongates (compresses) the nerve modulation assembly 104 and releasing the pull wire, releases the force on the nerve modulation assembly 104, expanding it. Moreover, a member to pull, push, or release the pull wire may be configured in the device's handle 216 allowing operators to easily expand or compress the nerve modulation assembly 104, as required. Alternatively, the actuation mechanism may be present at the proximal end 210 of the elongate member 208.

Where nerve modulation assembly 104 is expanded by some external force, the nerve modulation assembly 104 does not expand on its own. Thus, an expanding mechanism may be required to impose an outward radial force on the modulation assembly 104 to expand it. Such expansion mechanism (not shown) may include balloons inflated by fluids, or dilators. Other such expansion mechanism may also be utilized without departing from the scope of the present disclosure. For example, springs or levers may be utilized to expand the nerve modulation assembly 104. Similarly, the nerve modulation assembly 104 itself may be formed of pivotal structures connected to one another. For instance, the modulation assembly 104 may be formed of multiple wires interconnected along pivotal joints. An outward force on the pivotal point expands the various wires connected to the point, expanding the nerve modulation assembly 104.

The expansion of the nerve modulation assembly 104 should be such that it does not cause damage to the artery by exerting a large force on the vessel walls. To prevent such large expansion diameters, the nerve modulation assembly 104 may include visualization features such as radiopaque struts or markers to visualize the extent of expansion using standard fluoroscopy methods. Further, the nerve modulation assembly 104 may include a force or expansion-limiting component that prevents the modulation assembly 104 from expanding beyond a certain limit. Often, the expansion limit may be set during manufacturing of the modulation assembly 104. For example, operators may know the average size of renal arteries, and they may ensure the basket does not expand beyond the average artery size. For example, the diameter of the expanded modulation assembly 104 may be maintained below about 4 French. The expansion-limiting component may be employed on both the inner and outer basket, as desired.

The following figures and description illustrate a specific exemplary configuration of the nerve modulation assembly 104.

FIG. 3A is a schematic illustrating a distal portion of the ablative catheter system 200 within a blood vessel in a patient's body. Here, the nerve modulation assembly 104, having a proximal end 304 a distal end 306, is in the expanded state. The nerve modulation assembly 104 generally forms a double basket, including an outer basket 308 and an inner basket 310. The outer basket 308 is longer than and encloses the inner basket 310 such that the surface of the inner basket 310 is spaced away from the vessel wall 302, thus never making contact with vessel wall 302. The inner basket 310 includes electrodes 312 positioned on its surface, as desired. The inner basket 310 may be longitudinally centered as shown in FIG. 3A or may be longitudinally offset with respect to the outer basket 308. The electrodes 312 may be centered on the inner basket 310 as shown in FIG. 3A or may be offset or angled on the inner basket 310.

The outer basket 308 includes multiple spacer struts 314 and the inner basket 310 includes multiple electrode struts 316. The struts 314, 316 are joined together along the longitudinal axis at their proximal and distal ends. In the illustrated embodiment, struts 314, 316 axially extend from the proximal end 304 to the distal end 306. In other embodiments, however, struts 314, 316 may follow a spiral or helical path from the proximal end 304 to the distal end 306. It will be understood that other basket 308, 310 configurations are also within the scope of the present disclosure. In addition, the number of struts 314, 316 constituting the inner basket 310 and outer basket 308 may vary, as desired. For example, the outer and inner baskets 308, 310 may include 5 struts each. In an aspect, the outer basket 308 may include 6 struts, while the inner basket 310 may include only 4 struts. These are just examples. It is contemplated that either the outer basket 308 or the inner basket 310 may have any number of struts 314, 316 desired.

Struts 314, 316 generally remain substantially parallel to the longitudinal axis in the compressed state, and radially expand in the expanded state. A center portion of struts 314 and 316 expand to form baskets. As shown, the outer basket 308 expands to a greater degree as compared to the inner basket 310, keeping the inner basket struts 316 spaced apart from the vessel walls 302.

Each strut 314, 316 may be formed of a single wire extending from the proximal end to the distal end. Alternatively, the struts 314, 316 may be formed of multiple wires twisted or braided along the length of the nerve modulation assembly 104. Moreover, the multi-wire struts 314, 316 may extend along the entire length of the retracting member and the sheath, or only the length of the retracting member. In other cases, portions of the struts 314, 316 may be formed of single wires, while other portions may be formed of multiple wires. In yet other cases, the thickness of the wires may be uniform along the length of the struts. Alternatively, the wires may be thicker in the middle and thinner at the proximal and distal portions of the struts 314, 316, or vice-versa.

Each strut 314, 316 may assume varying shape and configuration. For example, struts 314, 316 may be round, flat ribbons, solid wires, or hollow tubes. In addition, all struts 314, 316 may be identical, or different struts 314, 316 may be shaped differently. If round struts are used, it may be desirable to bias the strut to expand in the desired direction by a forming method or localized plastic deformation. Flat ribbons may have a width (tangent to the circumference of the device) greater than the thickness (the dimension along a radius) to ensure bowing in the proper direction when expanded. Alternatively, a predetermined bias may be built into the strut 314, 316.

In general, spacer struts 314 may be made any suitable insulative material acting as electrical spacers. Struts 316, however, may be made of a conductive material with an insulative cladding. A portion of the struts 316 may be bare wire acting as electrodes 312. For example, a center portion of the struts 316 may be without a cladding, while all other portions may have the insulative cladding. Alternatively, the struts 316 may also be made of completely insulative material and external electrodes 312 may be attached to portions of the nerve modulation assembly 104. For example, one or more wireless or wired electrodes connected to the power source may engage with the one or more struts 316.

FIG. 3B is a schematic of a cross-section of the nerve modulation assembly 104 of FIG. 3A showing the electrodes 312 and the spacer struts 314. In FIG. 3B, the struts 314 of the outer basket 308 have been connected with a circular line and the struts 316 of the inner basket 310 have also been connected with a circular line to illustrate the outer profile of both the outer and inner baskets 308, 310. However, this is merely exemplary. The struts 314, 316 are not necessary interconnected. Further, diagonal lines connecting outer struts 314 offset from inner struts 316 have been included to illustrate the struts 314, 316 may be offset from one another, although this is not required. When fully expanded, the spacer struts 314 may contact or nearly contact the vessel wall 302, while electrodes 312 positioned on the inner basket 310 confined within the outer basket 308, preventing contact between vessel walls 302 and electrodes 312.

Further, the electrodes 312 may each be connected to a power supply, such as power source 218, such that each electrode may be operated separately and current may be maintained to each electrode 312. The power source may activate each electrode 312 one at a time. The next electrode is activated only after a first electrode is activated and deactivated. Alternatively, the electrodes 312 may be activated simultaneously.

When electrical signals are passed through the struts 316, the bare portions behave as electrodes 312. Therefore, based on the required number and position of electrodes 312, portions of the nerve modulation assembly 104 may be left bare.

Electrodes 312 may be positioned on struts 316 in any suitable manner, designed to provide ablative RF energy to selected areas adjacent the target vessel. In some embodiments, all electrodes 312 may be positioned on the center portion of each internal strut 316. Alternatively, electrodes 312 may be staggered so that all the electrodes 312 are not located at the same axial level. Such an arrangement may allow electrodes 312 to target different ablation sites. For example, the electrode 312 for one strut 316 may be in the central portion, for another strut 316 may be in the proximal portion, and for a third strut 316 may be in the distal portion. In addition, the number of electrodes 312 on struts 316 may vary. In an embodiment, only one of the struts 316 may include bare electrodes 316. Alternatively, some or all of the struts 316 may include the electrodes 312. Different alternatives of the electrodes 312 may be contemplated. For example, bare electrode portions 312 on struts 316 may be identically or differently shaped such as round or oblong paddles.

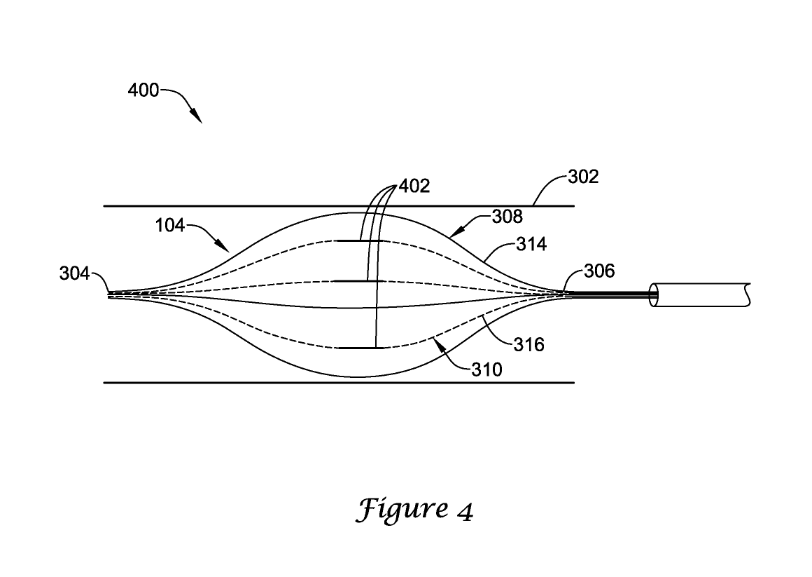

FIG. 4 illustrates an alternate embodiment of the ablative catheter system 400 depicting the nerve modulation assembly 104 deployed within the blood vessel 302. The nerve modulation assembly 104, extending from the distal end of the sheath 106, is configured to assume an expanded configuration.

A number of elements of ablative system 400 are similar to those shown in FIG. 2 such as the outer basket 308, inner basket 310, and struts 314, 316. Here, the ablative catheter system 400 includes a wider electrode 402 (as compared to the struts 316), as opposed to system 200, where the electrodes 312 are bare wires having a cross-section smaller than the remaining strut portion. In the illustrated embodiment, the electrodes 402 may be oblong, paddle, or suitably shaped having a cross-section wider than the proximal portion of the struts 316.

The rigidity and characteristics of the material used to form the nerve modulation assembly 104 determine expandability of the nerve modulation assembly's 104. For example, the thickness of the material may vary between the central portion, and the distal and proximal portions, causing the central portion to deviate greater than the proximal and distal portions. In addition, the outer and inner struts 314, 316 may expand to a different degree. For example, the spacer struts 314 may expand more so than the electrode struts 316, creating spaces between the electrode struts 316 and the vessel wall 302. To this end, the material composition may vary between the central and end portions of each strut and between spacer and electrode struts 314, 316, varying the expandability of these portions. In some embodiments, stainless steel may be used to form one portion, while tungsten, platinum, palladium, or a suitable polymer may be used to form other portions. Other techniques to vary the expandability of the struts may be employed just as easily, as understood by those of skill in the art.

Further, the degree of expansion, the materials used, and the thickness of the struts 314, 316 may vary within the struts without departing from the scope of the present disclosure. Moreover, different levels of expansion may be carried out for the different inner struts 316 so that the electrodes 312 are at a varied distance from the artery walls.

It will be understood that other variations in configuration are possible as long as the nerve modulation assembly 104 includes insulated portions in contact with the vessel wall and bare electrode portions 312 away from the vessel wall. For example, the nerve modulation assembly 104 may be made of expandable conductor wires shaped as an ellipse or a circle. The elliptical or circular member may be stored in a compressed state within the sheath 106, and when the nerve modulation assembly 104 is actuated to extend beyond the distal end 206 of the sheath 106 the nerve modulation assembly 104 may expand. In this type of nerve modulation assembly 104, the electrodes 312 may be positioned at the distal or proximal end of the nerve modulation assembly 104. Alternatively, the inner struts may have a zigzag shape, bends, or bumps to position the electrodes 312 as desired.

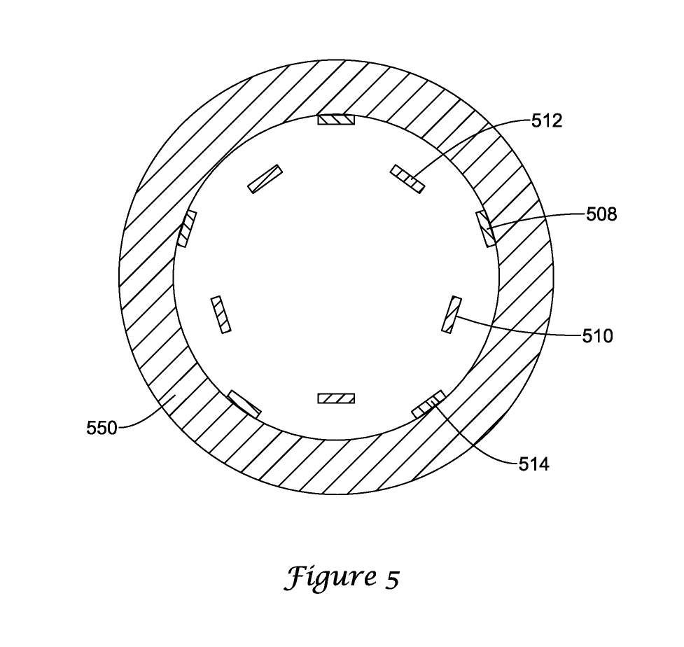

For example, FIG. 5 is a cross-sectional view of an example system disposed in an expanded state within a blood vessel 550. The outer basket 508 comprises five spacer struts 514 that have a ribbon-shaped cross-sectional profile. An inner basket 510 comprises five electrode struts 512 that also have a ribbon-shaped cross-sectional profile. The electrode struts 512 of the inner basket 510 are offset from the spacer struts 514 of the outer basket, although this is not required. Furthermore, while the system is described as including five space struts 514 and five electrode struts 512, it is contemplated that there may be any number of struts desired in either the outer basket 508 or the inner basket 510. Additionally, the dimensions illustrated in FIG. 5 are merely examples. The struts 512, 514 may take any shape and/or size desired. Similarly, the spacing between the vessel wall 550 and the electrode struts 512 may be any distance desired. The embodiment is otherwise similar to that described with respect to FIGS. 3A and 3B.

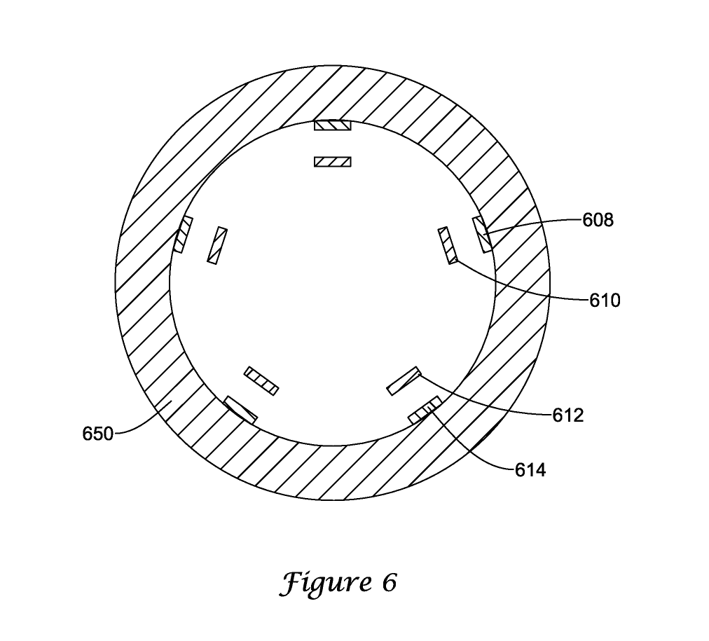

FIG. 6 is a cross-sectional view of an example system disposed in an expanded state within a blood vessel 650. The outer basket 608 comprises five spacer struts 614 that have a ribbon-shaped cross-sectional profile. An inner basket 610 comprises five electrode struts 612 that also have a ribbon-shaped cross-sectional profile. The electrode struts 612 of the inner basket 610 are in line with the spacer struts 614 of the outer basket, although this is not required. Furthermore, while the system is described as including five space struts 614 and five electrode struts 612, it is contemplated that there may be any number of struts desired in either the outer basket 608 or the inner basket 610. Additionally, the dimensions illustrated in FIG. 6 are merely examples. The struts 612, 614 may take any shape and/or size desired. Similarly, the spacing between the vessel wall 650 and the electrode struts 612 may be any distance desired. The embodiment is otherwise similar to that described with respect to FIGS. 3A and 3B.

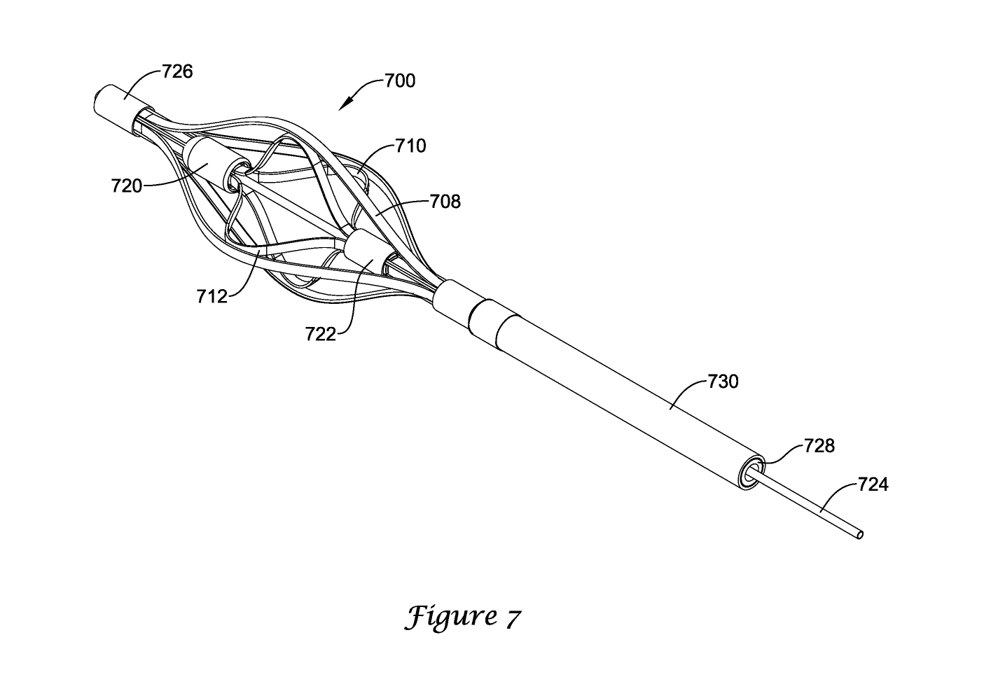

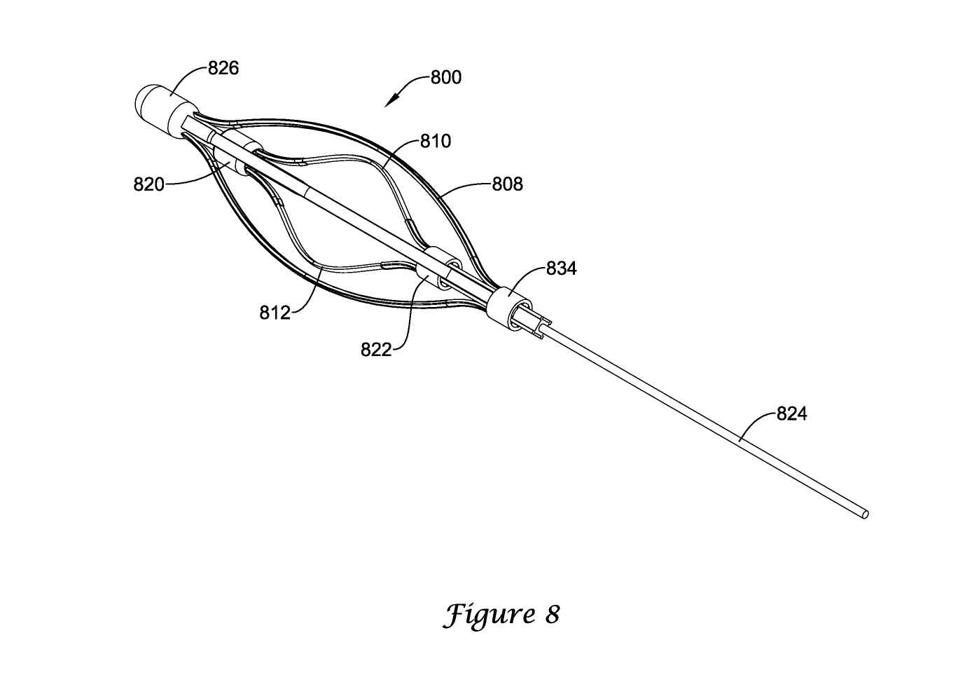

FIGS. 7 and 8 illustrate a portion of example ablative catheter systems with an ablative member in the expanded state. Systems 700 and 800 each have inner baskets 710,810 and outer baskets 708,808 that comprise struts having a ribbon profile. The expandable portion of inner basket 710 of system 700 is confined by a distal ring 720 and a proximal ring 722. Both rings 720 and 722 are within the outer basket 708. A pull wire 724 has a distal stop 726 and is freely slidable within a lumen 728 of the system 700. When the pull wire is moved proximally relative to a catheter 730 attached to the baskets, the baskets 708, 710 are moved to the expanded shape shown in FIG. 7. In the embodiment of FIG. 8, the pull wire 824 is fixed to the distal end 826 of the system 800. A proximal end 834 of the outer basket 808 is fixed to a catheter (not shown) that extends proximally over the pull wire 824. Relative proximal movement of the pull wire 824 relative to the catheter causes the baskets 808,810 to expand to the expanded configuration shown in FIG. 8. Distal and proximal stops 820,822 on the inner basket 810 within the outer basket 808 cause the radial expansion of the inner basket to be less than that of the outer basket. The electrode struts 712, 812 of the systems 700, 800 are electrically connect to a power source.

The active portions of the electrode struts 712, 812 may vary. For example, the whole of a strut 712 from ring 720 to 722 may be bare and act as an electrode or only a portion of the strut may be bare and act as an electrode. The non-electrode portions are coated with an electrically insulating material. A proximal portion, middle portion or distal portion may be the active electrode portion. In some embodiments, only an inner portion of the strut is the active electrode portion and the outer surface (and, in some embodiments, the edges between the inner and outer surfaces) are electrically insulated. In some of these embodiments as well, only a portion of the inner surface is the active electrode portion. For example, in one embodiment, the active electrode portion is the middle portion of the inner surface and the remainder of the strut 712 is insulated. It can be appreciated that the electrode struts of subsequent embodiments may readily include these variations discussed herein.

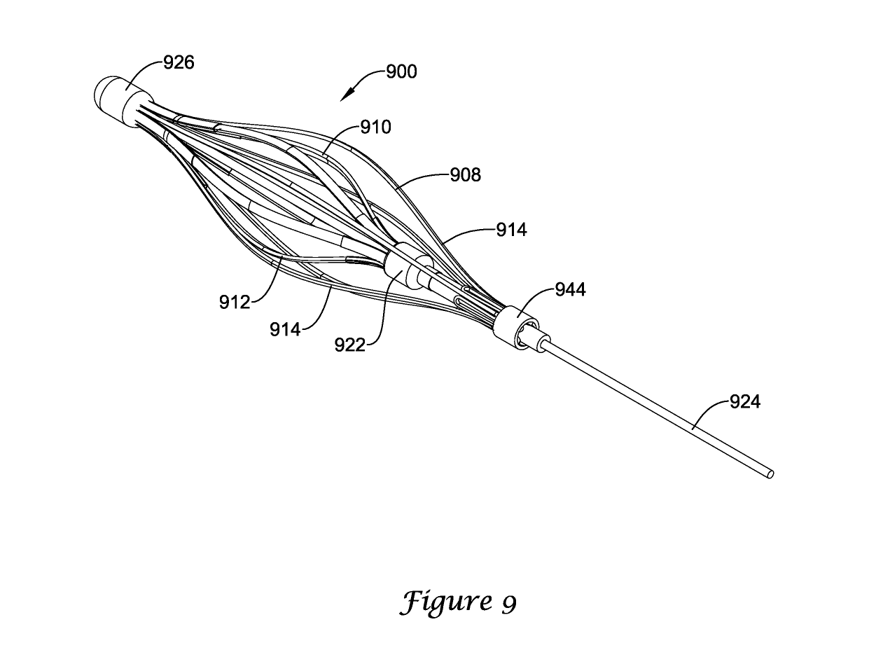

FIG. 9 illustrates a portion of an ablative catheter system 900 with an ablative member in the expanded state. In some instances, the inner basket 910 and outer basket 908 of system 900 may be formed from the same tubular precursor by a plurality of longitudinal slots cut in the tubular precursor. It is contemplated the size of the longitudinal slots may be varied to achieve the desired basket shape. In other instances, the inner basket 910 may be formed from a first tubular precursor and the outer basket 908 may be formed from a second tubular precursor. It is contemplated that the inner basket 910 may be formed from a smaller tubular precursor than the outer basket 908, although this is not required. The proximal ends of the five struts 912 of the inner basket 910 are joined by a fixation element 922 that is distal a fixation element 944 that joins the proximal ends of the five struts 914 of the outer basket 908. While the inner and outer baskets 910, 908 are described as including five struts 912, 914, it is contemplated that the baskets 908, 910 may include any number of struts desired. Elements 922 and 944 are fixed relative to each other and slide over a pull wire 924 that is fixed to the distal end 926 of the system 900. Element 944 is further fixed to a catheter (not shown) that extends proximally over the pull wire 924. The electrode struts 912 of the inner basket 910 are electrically connected to a power source. The spacer struts 914 of the outer basket 908 are electrically insulated. The system may be biased to a closed state such that pulling on the pull wire 924 expands the system or biased to an open state such that pushing on the pull wire 924 collapses the system from an expanded state.

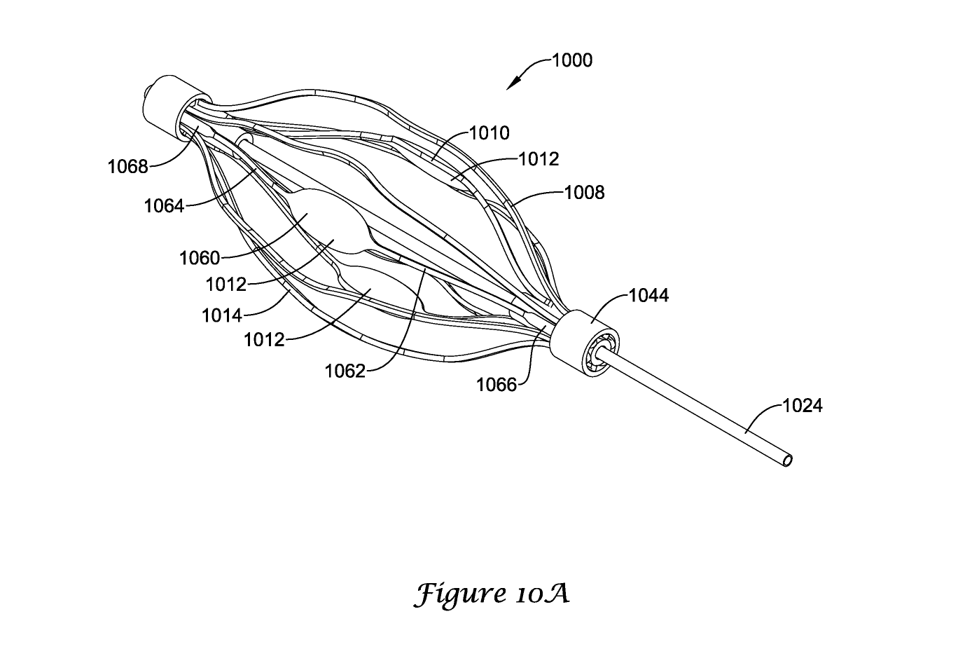

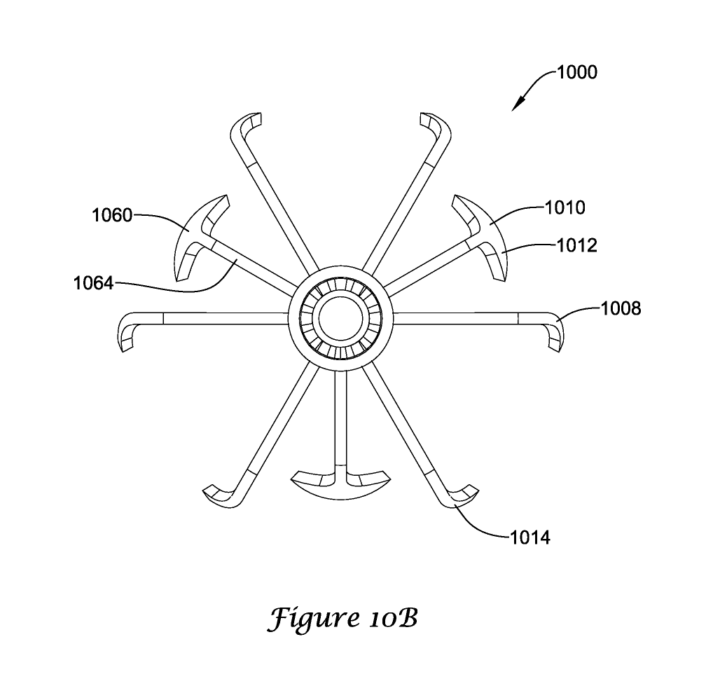

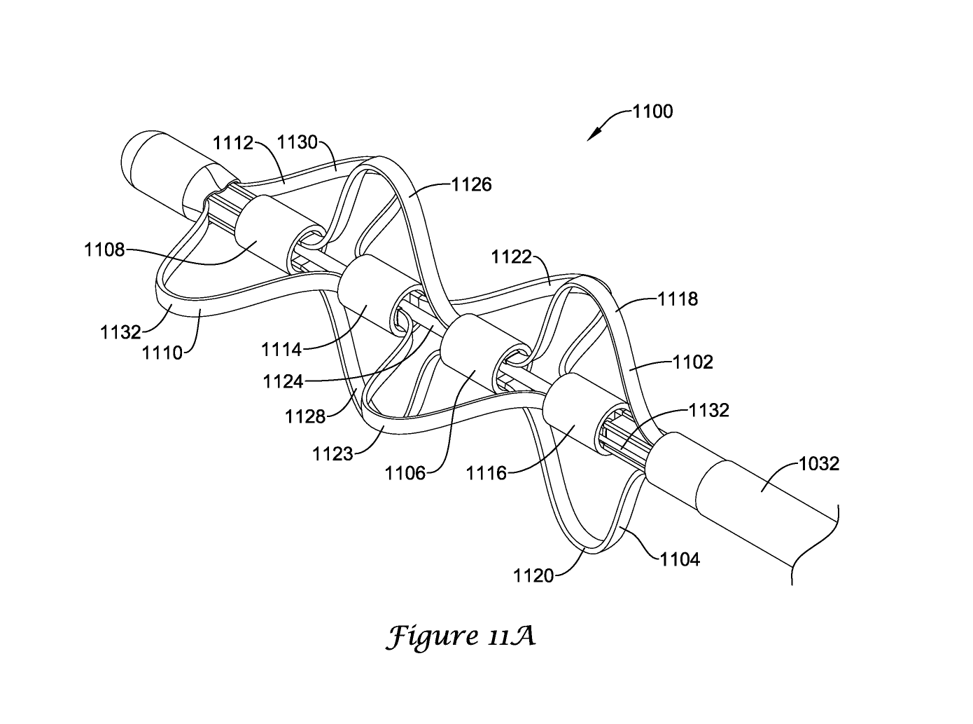

FIGS. 10A and 10B illustrate an isometric and an end view, respectively, of the distal portions of an ablative catheter system 1000 that is similar to system 900 except as otherwise noted. The system 1000 may include an inner basket 1010 and an outer basket 1008. The inner basket 1010 may include, but is into limited to, three electrode struts 1012 that have wider active electrode portions 1060. The proximal strut portion 1062 and distal strut portion 1064 of each electrode strut 1012 may be insulated such that portion 1060 is the only active portion of the electrode strut. Each electrode strut 1012 may also include a stiffer proximal base portion 1066 and stiffer distal base portion 1068 that exhibit greater resistance to the compressive bending force of the pull wire 1024. The greater stiffness of portions 1066, 1068 may be imparted by additional material or a different cross-sectional profile. Pull wire 1024 is fixed to the distal end of the device and relative movement between the pull wire 1024 and the proximal end 1044 of the baskets 1008, 1010 may cause expansion of the device. The outer basket 1008 may include, but is not limited to, six spacer struts 1014. A central portion of each spacer strut 1014 is bent away from the nearest electrode portion 1060, as illustrated in FIG. 10B. The system may be biased to a closed state such that pulling on the pull wire 1024 expands the system or biased to an open state such that pushing on the pull wire 1024 collapses the system from an expanded state.

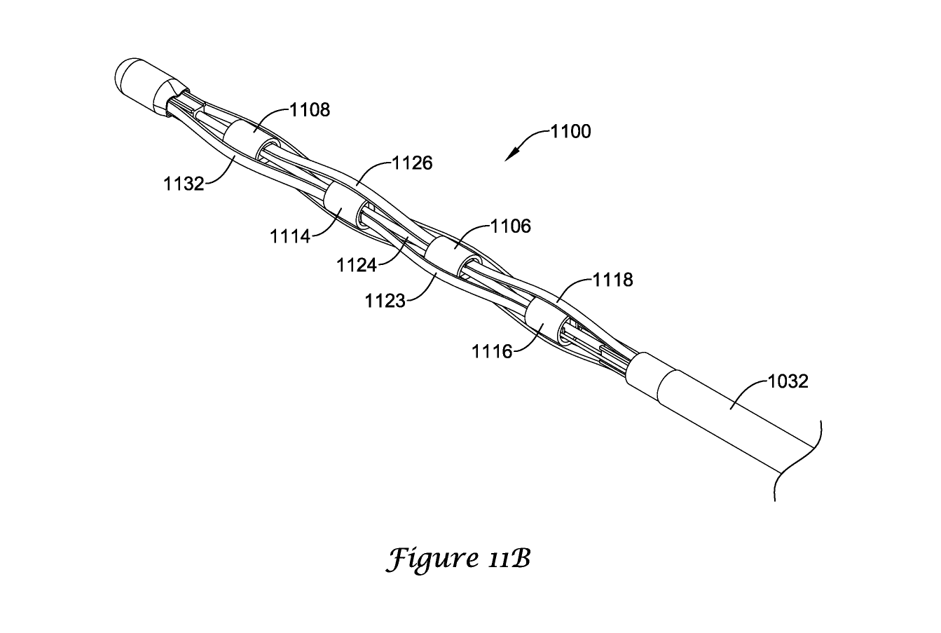

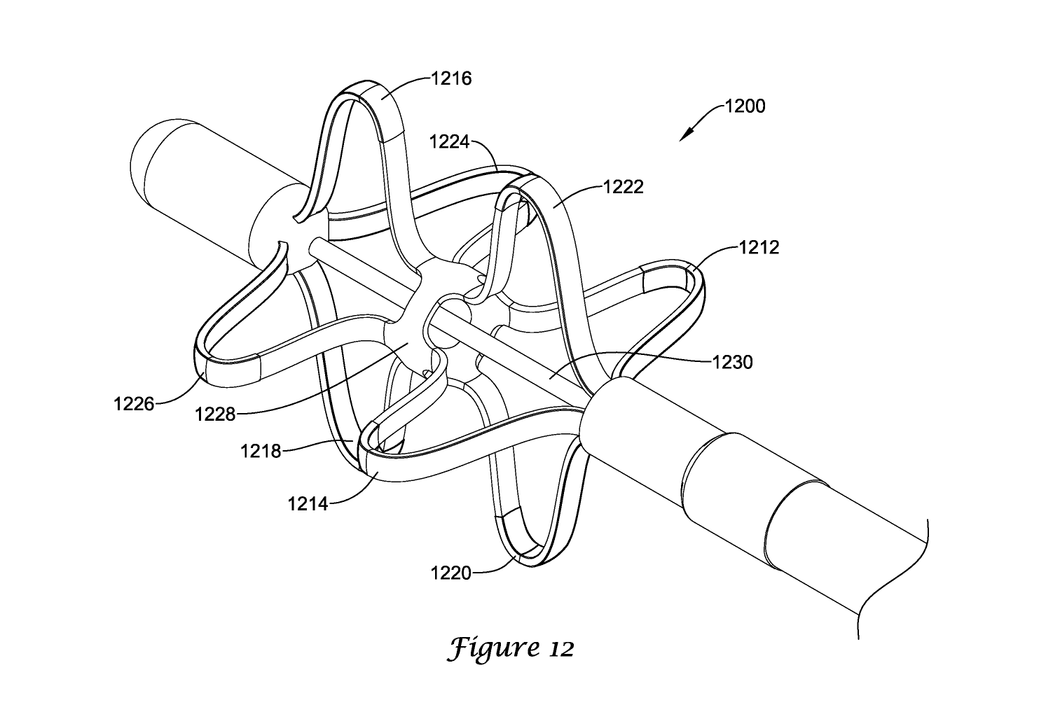

FIGS. 11A and 11B are isometric views of the distal portion of an ablative catheter system 1100 shown in an expanded state and a collapsed state, respectively. System 1100 is an ablative system where the electrodes may contact the vessel wall. A pull wire 1124 is fixed to the distal end of the system and proximal movement of the pull wire 1124 relative to the proximal end of the system causes expansion. A first pair of struts 1102 and 1104 is fixed proximally and distally and by rings 1106, 1108. In some instances, the first pair of struts 1102, 1104 may be positioned generally opposite from one another. For example, the first strut 1102 may be configured to contact the vessel wall at a first location and the second strut 1104 may be configured to contact the vessel wall approximately 180° from the first location. A second pair of struts 1110 and 1112 is likewise fixed proximally and distally and by rings 1114, 1116. In some instances, the second pair of struts 1110, 1112 may be positioned generally opposite from one another. For example, the third strut 1110 may be configured to contact the vessel wall at a first location and the fourth strut 1112 may be configured to contact the vessel wall approximately 180° from the first location. Thus, when the system is expanded, alternating apexes 1118, 1120, 1122, 1123, 1126, 1128, 1130 and 1132 are created. Apexes 1120, 1122, 1126 and 1132 are insulated and thus do not act as electrodes. Apexes 1118, 1123, 1128 and 1130 are bare and thus act as electrodes. The pattern of bare apexes forms a helical pattern with an active electrode approximately every 90 degrees. It can be appreciated that the profile of the strut 1102, 1104, 1110, 1112 at the electrode apexes may be altered if desired. For example, the apexes may be shaped as portions 1060 of FIGS. 10A and 10B. Conductors 1132 (not all illustrated) provide power to the struts 1102, 1104, 1110, 1112 and a hollow catheter 1032 extends proximally over the pull wire 1124. The system may be biased to a closed state such that pulling on the pull wire 1124 expands the system or biased to an open state such that pushing on the pull collapses the system from an expanded state.

The system 1200 shown in FIG. 12 is similar to that of system 1100 except that the struts may be formed from a single tubular member and the electrode portions of the struts form a more compact ablation pattern. In some embodiments, apex portions 1212, 1214, 1216, 1218 may be insulated while apex portions 1220, 1222, 1224, 1226 are bare and thus able to act as electrodes. In other embodiments, the apex portions 1212, 1214, 1216, 1218, 1220, 1222, 1224, 1226 are all insulated while the area of the tubular member (proximate to and including waist 1228) may be bare and able to act as electrodes. In a contemplated variation, the struts are formed separately and attached to a central ring (corresponding to waist 1228). The electrode apexes 1212, 1214, 1216, 1218, 1220, 1222, 1224, 1226 may also be changed from the ribbon profile shown. For example, they may be shaped like portions 1060 of FIG. 10A. The struts are fixed proximally to a tubular member and distally to the distal end of the system. A pull wire 1230 is likewise fixed to the distal end and slidable within the tubular member. The system may be biased to a closed state such that pulling on the pull wire 1230 expands the system or biased to an open state such that pushing on the pull wire 1230 collapses the system from an expanded state.

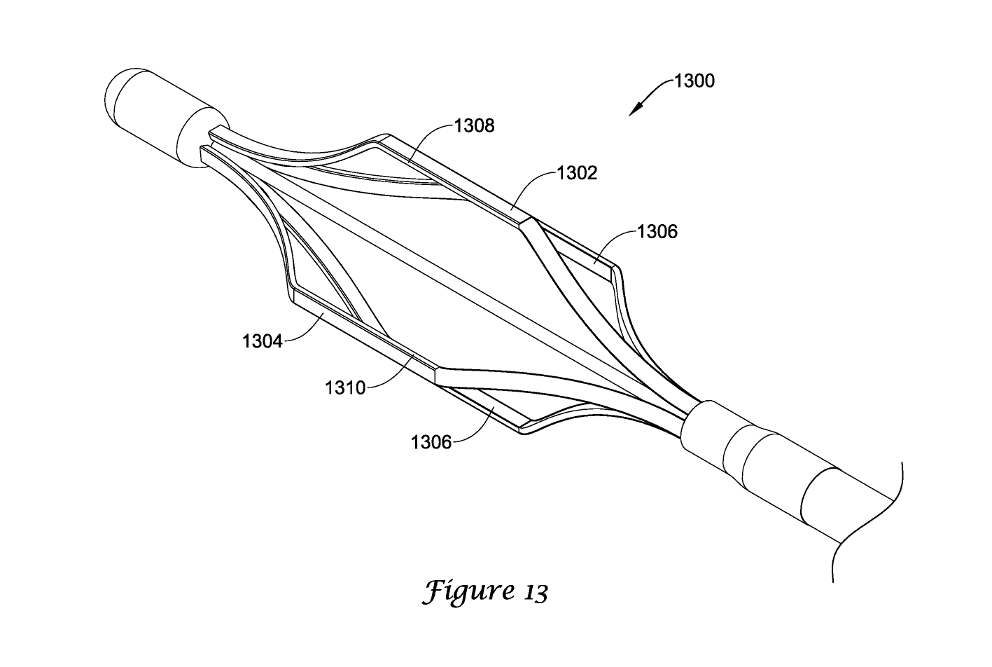

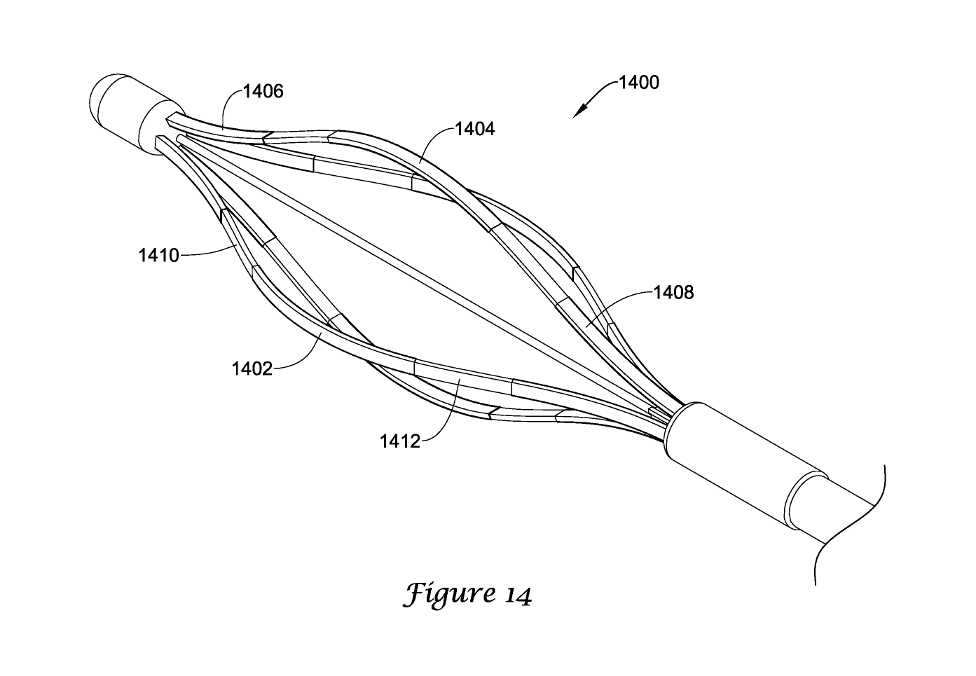

FIGS. 13 and 14 are isometric views of example embodiments of non-contact ablative catheter systems. The term “non-contact” is meant to signify that no active or electrically emitting portion of the electrode touches a vessel wall when the system is properly used in a blood vessel of conventional shape. Each system includes struts that are expanded by the use of a pull wire. The systems may be biased to a closed state such that pulling on the pull wire expands the system or biased to an open state such that pushing on the pull wire collapses the system from an expanded state. The pull wire and the struts are fixed together at their distal ends. The struts are fixed to a tubular member through which the pull wire slides at their proximal ends. The struts may have a uniform cross-section such as the illustrated flat ribbon or may have another desired shape. For example, the struts may widen at the active electrode portions. The struts may further be shaped to expand in a particular manner. For example, the struts of FIG. 13 are illustrated as having a flat central section when expanded. The struts may be altered to have the football shaped expansion profile of FIG. 14, for example.

In system 1300 of FIG. 13, each strut 1302 has an outer face 1304 that faces radially outwardly, an inner face 1306 that faces radially inwardly and may include two side faces 1308, 1310 that join the inner and outer faces. The outer face 1304 and the two side faces 1308, 1310 of each strut are covered with an electrically insulating material. The inner face 1306 is free from the electrically insulating material and is thus free to act as an electrode. In some instances, the inner face 1306 may be 100% free from insulating material. In other instances, the inner face 1306 may be partially covered with insulating material. For example, the inner face 1306 may be approximately 90% free from insulating material, approximately 80% free from insulating material, approximately 70% free from insulating material, approximately 60% free from insulating material, approximately 50% free from insulating material, approximately 40% free from insulating material, approximately 30% free from insulating material, approximately 20% free from insulating material, or approximately 10% free from insulating material. These are just examples. In some embodiments, the electrically insulating material covers the outer face 1304 and contiguous portions of the side faces 1308, 1310 while portions of the side faces 1308, 1310 contiguous with the inner face 1306 are bare. In some embodiments, the distal and proximal portions of the inner face 1306 are also covered with an electrically insulating material.

In system 1400, each strut 1402 includes an apex portion 1404 that is electrically insulated and a distal base portion 1406 and a proximal base portion 1408 that are also electrically insulated. Each strut 1402 also includes bare portions 1410 and 1412 that are free from insulating material and thus can act as electrodes. Each bare portion 1410,1412 is spaced from the center of the apex portion 1404 and is thus kept spaced from a vessel wall when system 1400 is expanded. In some embodiments, the inner face of the apex portion 1404 is free from insulating material or is only partially insulated so that the inner face of the apex portion 1404 may act as an electrode as well. In some instances, the inner face may be 100% free from insulating material. In other instances, the inner face may be approximately 90% free from insulating material, approximately 80% free from insulating material, approximately 70% free from insulating material, approximately 60% free from insulating material, approximately 50% free from insulating material, approximately 40% free from insulating material, approximately 30% free from insulating material, approximately 20% free from insulating material, or approximately 10% free from insulating material. These are just examples.

It is further contemplated that the size of the apex portion 1404 may vary depending on the desired application. In some instances, an outer surface of the apex portion 1404 may comprise at least 20% of the outer surface of the strut 1402. In other instances, the outer surface of the apex portion 1404 may comprise at least 30%, at least 40%, at least 50%, or at least 60%, of the outer surface of the strut 1402. These are just examples. In some embodiments, the outer surface of the apex portion 1404 may comprise no more than 60% of the outer surface of the strut 1402

To monitor the temperature of the any of the electrodes herein and the blood vessel walls, one or more sensors, such as temperature sensors, may be placed at different portions of the nerve modulation assembly 104. For instance, one sensor may be placed near the electrode to monitor electrode fouling or electrode temperature, and another sensor may be placed in the portion contacting the vessel wall to measure the temperature at the blood vessel. External devices connected to the sensors may be configured to raise alerts if any of the sensors detect temperatures over a preconfigured threshold value. If an alert is raised, operators may discontinue ablation or reduce power until the temperature at the electrode or at the vessel wall returns under the threshold value. Alternatively, operators may simply monitor the temperatures and discontinue operation when temperatures exceed a certain value. In an alternate embodiment, the impedance of the electrodes may be measured by the control and power element to monitor the procedure.

The shape of nerve modulation assembly 104 described in the present disclosure may eliminate the possible problems associated with an electrode touching the artery walls and causing injury there. Further, being spaced from the vessel walls, the electrode may circumferentially radiate RF energy, equally ablating the nerves surrounding the artery. It may be preferred to space the electrodes as close as possible to the vessel wall without actually touching the vessel wall with the bare metal of the electrodes. Such a configuration may minimize the power requirements of the device while reducing or eliminating excessive heating of deeper surrounding tissues.

In use, any of the systems may be introduced percutaneously as is conventional in the intravascular medical device art. For example, a guidewire may be introduced percutaneously through a femoral artery and navigated to a renal artery using standard radiographic techniques. The catheter sheath 106 may be introduced over the guide wire and the guide wire may be withdrawn. The elongate member and the ablative member may then be introduced in the sheath 106 and urged distally to the desired location. Once there, the sheath may be retracted proximally to allow the ablative member to expand or the ablative member may be urged distally to extend beyond the distal end of the sheath.

The outer and inner basket may be actuated simultaneously or actuated separately. In one embodiment, once the nerve modulation assembly 104 extends from the sheath 106, both the inner and outer basket may expand to their desired configuration. Alternatively, the outer basket may be actuated first so that the outer basket may snuggly fit with the vessel walls. The inner basket may then be actuated based on the configuration of the outer basket, ensuring that the degree of expansion of the inner basket is less than the outer basket.

The electrodes may then be activated to ablate nerve tissue. During this procedure, the ablative member may continuously monitor the impedance and/or temperature at the electrodes and the vessel walls. Further, the electrodes may be activated sequentially or simultaneously, as desired. Radiography techniques may be utilized to monitor the tissue being ablated. Once the tissue is sufficiently ablated, the catheter sheath may be advanced or the ablative member may be retracted to compress the ablative member and retrieve it from the patient's body. Alternatively, the ablative member may be repositioned to perform further ablative procedures as desired.

Those skilled in the art will recognize that the present disclosure may be manifested in a variety of forms other than the specific embodiments described and contemplated herein. Accordingly, departure in forma and detail may be made without departing from the scope and spirit of the present disclosure as described in the appended claims.

What is claimed is:

1. A system for nerve modulation, comprising:
an elongate shaft having a longitudinal axis, a proximal end, a distal end, and a nerve modulation assembly disposed at the distal end, the nerve modulation assembly having a collapsed configuration and an expanded configuration;
wherein the nerve modulation assembly includes an inner basket and an outer basket;
wherein the inner basket includes a proximal end and a distal end, the inner basket including a plurality of electrode struts joined to each other at the proximal end of the inner basket and extending to the distal end of the inner basket, wherein each electrode strut includes an electrode;
wherein the outer basket includes a proximal end and a distal end, the outer basket comprising a plurality of spacer struts joined to each other at the proximal end of the outer basket and extending to the distal end of the outer basket;
wherein the inner basket and the outer basket are disposed at the distal end of the elongate shaft; and
wherein when the nerve modulation assembly is in the expanded configuration the plurality of spacer struts extend further radially outward from the longitudinal axis of the shaft than the plurality of electrode struts.

2. The system of claim 1, wherein the distance between the proximal and distal ends of the inner basket is less than the distance between the proximal and distal ends of the outer basket.

3. The system of claim 1, wherein each of the electrode struts comprise a conductive inner core and a layer of insulation disposed over the conductive inner core, and wherein the electrode is a portion of the electrode strut free from insulation.

4. The system of claim 1, wherein the electrode has a smaller cross-sectional profile than the remaining portion of the electrode strut.

5. The system of claim 1, wherein the electrode has a larger cross-sectional profile than the remaining portion of the electrode strut.

6. The system of claim 1, wherein the plurality of spacer struts comprises a non-conductive material.

7. The system of claim 1, wherein each of the plurality of spacer struts comprises an inner member surrounded by an insulating layer.

8. The system of claim 1, wherein the plurality of electrode struts are formed from a first single tubular precursor that is cut to define the electrode struts.

9. The system of claim 8, wherein the plurality of spacer struts is formed from a second single tubular precursor different than that of the first tubular precursor.

10. The system of claim 1, wherein the plurality of electrode struts and the plurality of spacer struts are both formed from a single tubular precursor.

11. The system of claim 1, further comprising a pull wire operably connected to a distal end of the nerve modulation assembly to move the nerve modulation assembly between the collapsed configuration and the expanded configuration.

12. A system for nerve modulation, comprising:
an elongate shaft having a proximal end, a distal end and a nerve modulation assembly at the distal end, the nerve modulation assembly including a basket configured to shift between a collapsed configuration and an expanded configuration;
wherein the basket has a proximal end and a distal end and comprising a plurality of inner struts and a plurality of outer struts;
wherein each of the inner struts include an electrode portion and an electrically insulated portion; and
wherein the basket is disposed at the distal end of the elongate shaft.

13. The system of claim 12, wherein the plurality of inner struts comprises a first strut, a second strut, a third strut and a fourth strut.

14. The system of claim 13, wherein the first strut is opposite the second strut and the third strut is opposite the fourth strut.

15. The system of claim 14, wherein the first and second struts are fixed together at a first position between the proximal and distal ends of the basket and the third and fourth struts are fixed together at a second position different from the first position between the proximal and distal ends of the basket.

16. The system of claim 14, wherein the first strut, second strut, third strut and fourth strut are fixed together at a single position between the proximal and distal ends of the basket.

17. The system of claim 12, wherein each of the inner struts forms a first apex defining the electrode portion and wherein each of the outer struts forms a second apex that is electrically insulated.

18. The system of claim 12, wherein each of the plurality of inner struts has an inner face and an outer face, and wherein the outer face is electrically insulated and at least a portion of the inner face is free of an insulating material.

19. The system of claim 12, further comprising a pull wire operably connected to the distal end of the basket to move the basket between the collapsed configuration and the expanded configuration.

20. A system for nerve modulation, comprising:
an elongate shaft having a proximal end and a distal end;
a basket assembly configured to move between a collapsed configuration and an expanded configuration disposed adjacent to the distal end of the elongate shaft, the basket assembly comprising:
an inner basket having a proximal end and a distal end and comprising a first plurality of struts, at least one of the first plurality struts including an electrode; and
an outer basket having a proximal end and a distal end and comprising a second plurality of struts, the second plurality of struts comprising an insulating material; and
wherein in the expanded configuration the outer basket has a cross-sectional profile larger than a cross-sectional profile of the inner basket.
